(19) United States
(12) Patent Application Publication
(10) Pub. No.: US 2012/0046511 A1
(43) Pub. Date: Feb. 23, 2012

(54) METHOD AND SYSTEM FOR SURFACE MODIFICATION OF SUPERADSORBENT MATERIAL FOR IMPROVED ENVIRONMENTAL AND URBAN AIR SAMPLING APPLICATIONS

(75) Inventors: Terence L. Schull, Alexandria, VA (US); Deborah E. Hunka, Belcamp, MD (US); Christopher L. Rector, Lee's Summit, MO (US); Michael J. Bowers, II, Sykesville, MD (US)

(73) Assignee: BAE Systems Information and Electronic Systems Integration Inc., Nashua, NH (US)

(21) Appl. No.: 13/183,495

(22) Filed: Jul. 15, 2011

Related U.S. Application Data
(60) Provisional application No. 61/365,010, filed on Jul. 16, 2010.

(51) Int. Cl. C07C 7/12

(52) U.S. Cl. 585/820

(57) ABSTRACT

A method for providing superadsorption of long chain hydrocarbon compounds using a material system comprising the steps of enhancing adsorption by decreasing reactivity at surface sites attractive to long chain hydrocarbon compounds and employing consequence management by maintaining a high rate of adsorptivity combined with high fidelity and accuracy of the material system.

### CROSS-REFERENCE TO RELATED APPLICATIONS

This Application claims rights under 35 USC §119(e) from U.S. Application Ser. No. 61/365,010 filed Jul. 16, 2010; and the U.S. application Ser. No. ______ (Atty. Docket BAEP-1291) filed Jul. 15, 2011, the contents both of which are incorporated herein by reference.

### BACKGROUND OF THE INVENTION

1. Field of the Invention

The present invention relates to providing enhanced desorption and more particularly to methods for providing enhanced desorption via due to decreased reactivity at surface sites attractive to long chain hydrocarbon compounds.

2. Brief Description of Related Art

Current superadsorbent materials do not provide adequate desorption of long chain hydrocarbon, i.e., alkane chains with 8 or more carbon atoms.

A need exists, therefore, for an improved method for providing enhanced desorption.

### SUMMARY OF THE INVENTION

The present invention is a method for providing superadsorption of long chain hydrocarbon compounds using a material system comprising the steps of:

(a) enhancing adsorption by decreasing reactivity at surface sites attractive to long chain hydrocarbon compounds; and
(b) employing consequence management by maintaining a high rate of adsorptivity combined with high fidelity and accuracy of the material system.

According to the present invention, the modification of the superadsorbent material leads to enhanced performance in desorption of the classes of long chain hydrocarbon, i.e., alkane chains with 8 or more carbon atoms, which in turn allows the for 1) the identification of the compounds in the original air sample and 2) the ability to correlate a relative concentration of the analytes to an original concentration. While the surface modification of the material allows for more polar compounds to be adsorbed, the desirable physical properties such as very high surface area and mass transfer rates of the superadsorbent material are retained.

### BRIEF DESCRIPTION OF THE DRAWINGS

The present invention is further described with reference to the following drawings wherein:

FIG. 1 is a graph showing modified and unmodified sorbent challenged with polar analytes in a preferred embodiment of the invention.

### DETAILED DESCRIPTION OF THE PREFERRED EMBODIMENTS

(1) Desorption—The disclosed system/materials offer a way to provide enhanced desorption via due to decreased reactivity at surface sites attractive to long chain hydrocarbon compounds. This modification allows for the reversible adsorption of large hydrocarbons which leads detection of ensembles of chemical components which retain a concentration profile representative of the air originally sampled.

(2) Consequence Management—The combined act of sampling the air in an environment and subsequently detecting the adsorbed samples is defined as consequence management. The prior art methods of performing this function do not have any solution that can adsorb a wide variety of volatile organic compounds and rapidly desorb it with very high fidelity and accuracy.

The high rate of adsorptivity combined with high fidelity and accuracy of the material system is the solution for consequence management.

The invention is further described with reference to the following working example:

### Example 1

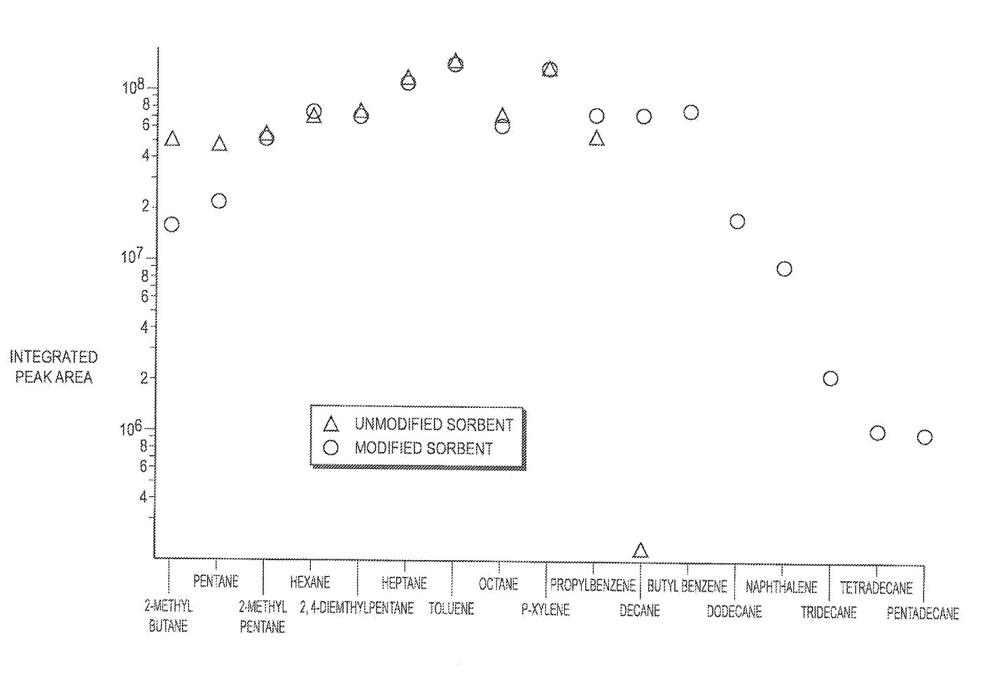

Results in FIG. 1 show the desorption results of an analyte mix with increasing numbers of carbons (C5-C15) from the unmodified sorbent and the sorbent modified using alkene thermografting to decrease the reactive step edge surface sites. Clearly, the modification increases the adsorbent's ability to desorb large hydrocarbons. In the unmodified case, the largest hydrocarbon detected was decane (C10), and in that case the desorption was minimal. The modification yields desorption all the way through the hydrocarbon series to pentadecane (C15).

What is claimed is:

1. A method for providing superadsorption of long chain hydrocarbon compounds using a material system comprising the steps of:
(a) enhancing adsorption by decreasing reactivity at surface sites attractive to long chain hydrocarbon compounds; and
(b) employing consequence management by maintaining a high rate of adsorptivity combined with high fidelity and accuracy of the material system.
